(19) United States
(12) Patent Application Publication
(10) Pub. No.: US 2023/0073205 A1
(43) Pub. Date: Mar. 9, 2023

(54) CONTAINER ASSEMBLY FOR COLLECTING BIOLOGICAL SPECIMENS

(71) Applicants: Sean Taesun Yeom, Cranbury, NJ (US); Ji Hyun Shin, I, Naju-Si (KR)

(72) Inventors: Sean Taesun Yeom, Cranbury, NJ (US); Ji Hyun Shin, I, Naju-Si (KR)

(21) Appl. No.: 17/988,439

(22) Filed: Nov. 16, 2022

Related U.S. Application Data
(63) Continuation-in-part of application No. 17/459,812, filed on Aug. 27, 2021.

(51) Int. Cl. A61B 10/00; B01L 3/00; G01N 1/31

(52) U.S. Cl. CPC A61B 10/0096; B01L 3/502; G01N 1/31; B01L 2300/047; B01L 2300/0832; B01L 2200/026; B01L 2300/042

(57) ABSTRACT

The present invention relates to a container assembly for collecting and transporting a tissue sample. The container includes a container having a chamber therein for receiving the tissue sample. The container assembly includes a cap configured to be attached to the container to close off the chamber. The cap includes a base section, which is configured to be attached to the container, and a movable section, which is movably coupled to the base section. The base section and movable section are configured to form a reservoir therebetween, the reservoir being sized and shaped so as to contain a preservation solution. The base section and the movable section are configured such that the preservation solution is discharged into the chamber from the reservoir when the movable section is moved relative to the base section from a closed position to an open position.

### CROSS-REFERENCE TO RELATED APPLICATION

This is a continuation-in-part of U.S. patent application Ser. No. 17/459,812 filed Aug. 27, 2021, the entire disclosure of which is incorporated herein by reference for all purposes.

### FIELD OF THE INVENTION

The present invention relates to collection container assemblies for collection of biological samples or specimens, such as biopsy samples.

### BACKGROUND OF THE INVENTION

Various collection containers have been developed in the past for collecting, storing and/or transporting biological specimens or samples from patients. The container assembly according to the present invention is adapted for collection of biological specimens or samples, including biopsy samples.

### SUMMARY OF THE INVENTION

In one embodiment, a container assembly for collecting and transporting a tissue sample is provided. In one embodiment, the container assembly includes a container having a chamber therein, the chamber being configured to receive the tissue sample. In one embodiment, the container assembly includes a cap configured to be attached to the container so as to close off the chamber. In one embodiment, the cap includes a base section, which is configured to be attached to the container, and a movable section, which is configured to be movably coupled to the base section. In one embodiment, the base section and movable section are configured to form a reservoir therebetween. In one embodiment, the reservoir is sized and shaped so as to contain a solution. In one embodiment, the base section includes at least one opening in fluid communication with the chamber when the cap is attached to the container. In one embodiment, the movable section is movable relative to the base section between a closed position, in which the movable section engages the base section so as to block off fluid communication between the reservoir and the at least one opening such that the solution is contained within the reservoir, and an open position, in which the reservoir is in fluid communication with the at least one opening such that the solution is discharged from the reservoir into the chamber through the at least one opening.

In one embodiment, the base section includes a first retaining member, in which the at least one opening is formed. In one embodiment, the movable section includes a second retaining member adapted to engage the first retaining member. In one embodiment, the first retaining member of the base section and the second retaining member of the movable section are configured to define the reservoir. In one embodiment, the second retaining member of the movable section is configured to engage the first retaining member of the base section in a liquid-tight manner so as to contain the solution in the reservoir when the movable section is positioned in its closed position, and to disengage, at least partially, from the first retaining member of the base section so as to allow fluid communication between the reservoir and the at least one opening and hence discharge of the solution from the reservoir to the chamber through the at least one opening when the movable section is positioned in its open position.

In one embodiment, the second retaining member of the movable section includes a cylindrical wall having an open bottom end. In one embodiment, the first retaining member includes a plug configured to close off the open bottom end of the cylindrical wall of the second retaining member.

In one embodiment, the cylindrical wall of the second retaining member of the movable section and the plug of the first retaining member of the base section are configured to define the reservoir. In one embodiment, the cylindrical wall of the movable section is configured to engage the plug so as to contain the solution in the reservoir when the movable section is positioned in its closed position. In one embodiment, the cylindrical wall is configured to disengage, at least partially, from the plug so as to allow the solution to discharge from the reservoir to the chamber through the at least one opening when the movable section is positioned in its open position.

In one embodiment, the at least one opening includes a plurality of discharge openings formed in the first retaining member. In one embodiment, the discharge openings are arranged so as to surround the plug.

In one embodiment, the movable section is rotatably coupled to the base section. In one embodiment, the movable section is movable relative to the base section in an axial direction in response to the rotation of the movable section relative to the base section.

In one embodiment, the movable section is movable in the axial direction away from the base section when the movable section moves from its closed position to its open position.

In one embodiment, the base section includes at least one first connector, while the movable section includes at least one second connector. In one embodiment, the at least one first connector is engageable with the at least one second connector so as to permit rotational movement of the movable section relative to the base section. In one embodiment, the base section includes at least one third connector, while the container includes at least one fourth connector. In one embodiment, the at least one third connector is engageable with the at least one fourth connector so as to movably attach the base section to the container. In one embodiment, each of the at least one first, second, third and fourth connectors includes at least one thread.

In one embodiment, a container assembly for collecting and transporting a tissue sample is provided. In one embodiment, the container assembly includes a container having a chamber therein. In one embodiment, the chamber is configured to receive the tissue sample. In one embodiment, a cap is attached to the container so as to close off the chamber. In one embodiment, the cap includes a base section, which is attached to the container, and a movable section, which is movably coupled to the base section. In one embodiment, the base section and movable section form a reservoir therebetween for containing a solution in the reservoir. In one embodiment, the base section includes at least one opening in fluid communication with the chamber. In one embodiment, the movable section is in a closed position, in which the movable section engages the base section so as to block off fluid communication between the reservoir and the at least one opening such that the solution is contained within the reservoir. In one embodiment, the movable section is movable to an open position, in which the movable section is, at least partially, disengaged from the base section so as to allow fluid communication between the reservoir and the at least one opening such that the solution can be discharged from the reservoir into the chamber through the at least one opening.

In one embodiment, a method for collecting a tissue sample is provided. In one embodiment, the method includes the steps of obtaining a container having a chamber therein; placing the tissue sample in the chamber of the container; obtaining a cap including a base section, which includes at least one opening, and a movable section, which is movably coupled to the base section, the movable section being positioned in a closed position, in which the movable section engages the base section and forms a reservoir containing a solution; placing the cap on the container so as to close off the chamber, the at least one opening being in fluid communication with the chamber; moving the movable section to an open position so as to disengage the movable section, at least partially, from the base section for allowing allow fluid communication between the reservoir and the at least one opening such that the solution is discharged from the reservoir into the chamber through the at least one opening.

### BRIEF DESCRIPTION OF THE DRAWINGS

In the drawing figures, which are not to scale, and where like reference numerals indicate like elements throughout the several views:

FIG. 1 is a top perspective view of a container assembly constructed in accordance with one embodiment;

FIG. 2 is a bottom perspective view of the container assembly shown in FIG. 1;

FIG. 3 is a front elevational view of the container assembly shown in FIG. 1, the right-side, left-side and rear views being identical to the view shown in FIG. 3;

FIG. 4 is a top plan view of the container assembly shown in FIG. 1;

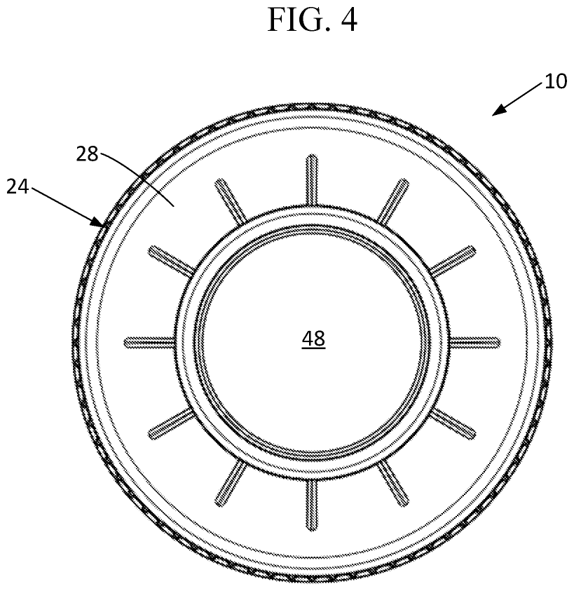

FIG. 5 is an exploded, top perspective view of the container assembly shown in FIG. 1;

FIG. 6 is an exploded, bottom perspective view of the container assembly shown in FIG. 1;

FIG. 7 is a view similar to FIG. 5, but looking at a different angle;

FIG. 8 is a cross-sectional view, taken along section line 8-8 and looking in the direction of the arrows, of the container assembly shown in FIG. 3, the container assembly having a cap which includes a reservoir shown in a closed condition in FIG. 8;

FIG. 9 is an exploded cross-sectional view of the container assembly shown in FIG. 8; and

FIG. 10 is a view similar to FIG. 8, except that the reservoir chamber is shown in an open condition.

### DESCRIPTION OF EXEMPLARY EMBODIMENTS

Embodiments are now discussed in more detail referring to the drawings that accompany the present application. In the accompanying drawings, like and/or corresponding elements are referred to by like reference numbers.

Various embodiments are disclosed herein; however, it is to be understood that the disclosed embodiments are merely illustrative of the disclosure that can be embodied in various forms. In addition, each of the examples given in connection with the various embodiments is intended to be illustrative, and not restrictive. Further, the figures are not necessarily to scale, and some features may be exaggerated to show details of particular components (and any size, material and similar details shown in the figures are intended to be illustrative and not restrictive). Therefore, specific structural and functional details disclosed herein are not to be interpreted as limiting, but merely as a representative basis for teaching one skilled in the art to variously employ the disclosed embodiments.

Subject matter will now be described more fully hereinafter with reference to the accompanying drawings, which form a part hereof, and which show, by way of illustration, specific examplary embodiments. Subject matter may, however, be embodied in a variety of different forms and, therefore, covered or claimed subject matter is intended to be construed as not being limited to any examplary embodiments set forth herein; exemplary embodiments are provided merely to be illustrative. Among other things, for example, subject matter may be embodied as methods, devices, components, or systems. The following detailed description is, therefore, not intended to be taken in a limiting sense.

Throughout the specification and/or claims, terms may have nuanced meanings suggested or implied in context beyond an explicitly stated meaning. Likewise, the phrase “in one embodiment” as used herein does not necessarily refer to the same embodiment and the phrases “in another embodiment” and “other embodiments” as used herein do not necessarily refer to a different embodiment. It is intended, for example, that covered or claimed subject matter include combinations of exemplary embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

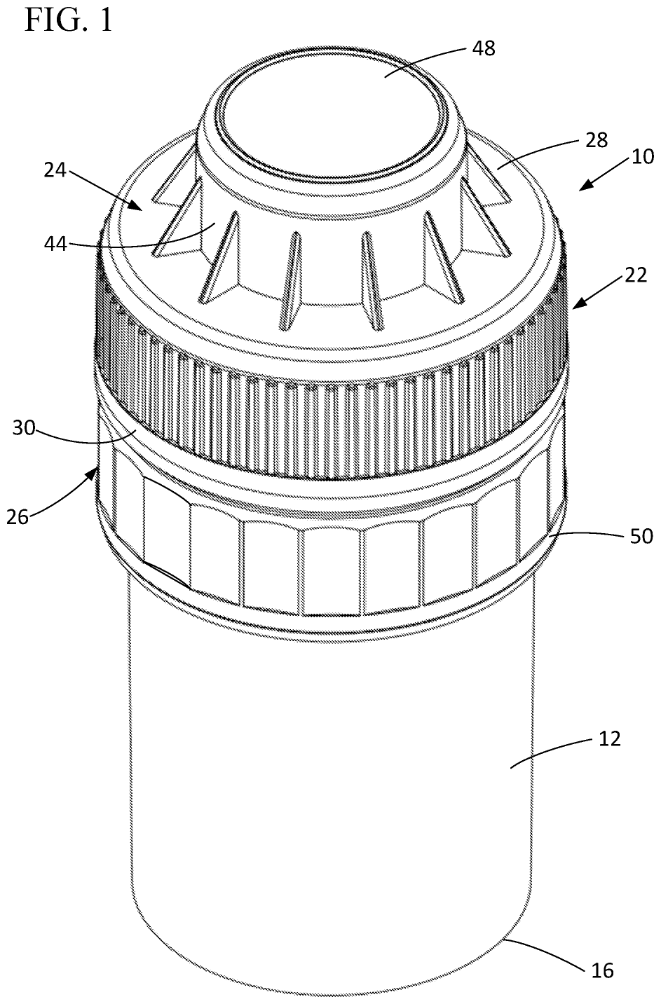

FIG. 1 illustrates a container assembly 10 constructed in accordance with an embodiment. In one embodiment, the container assembly 10 is adapted for collection, storage and/or transportation of a biological sample or specimen obtained from a patient. In other embodiments, the container assembly 10 is especially suited for collection, storage and/or transportation of a biopsy sample in a preservation solution (e.g., formalin), which can be harmful to a user of the container assembly 10 (e.g., a nurse performing the biopsy, etc.) if it or its fume is inhaled by or otherwise comes in contact with the user. In one embodiment, the container assembly 10 includes a reservoir in which the solution is pre-stored within the container assembly 10 and which is capable of safely releasing the solution into a biopsy storage chamber in the container assembly 10. In other embodiments, the container assembly 10 is adapted for use in collecting, storing and/or transporting other biological samples, either with or without a solution.

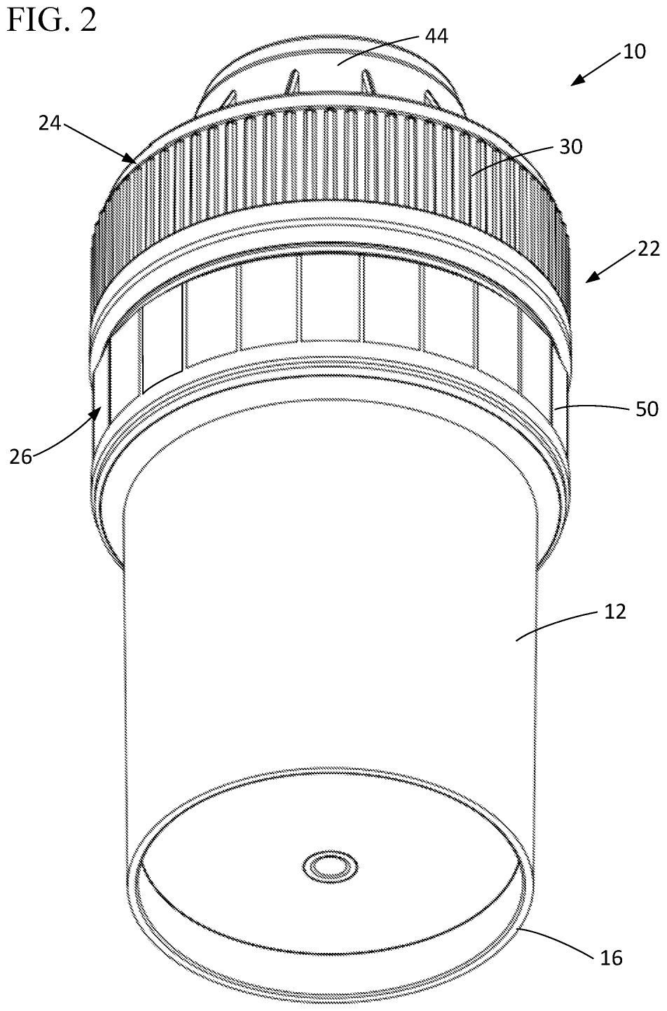

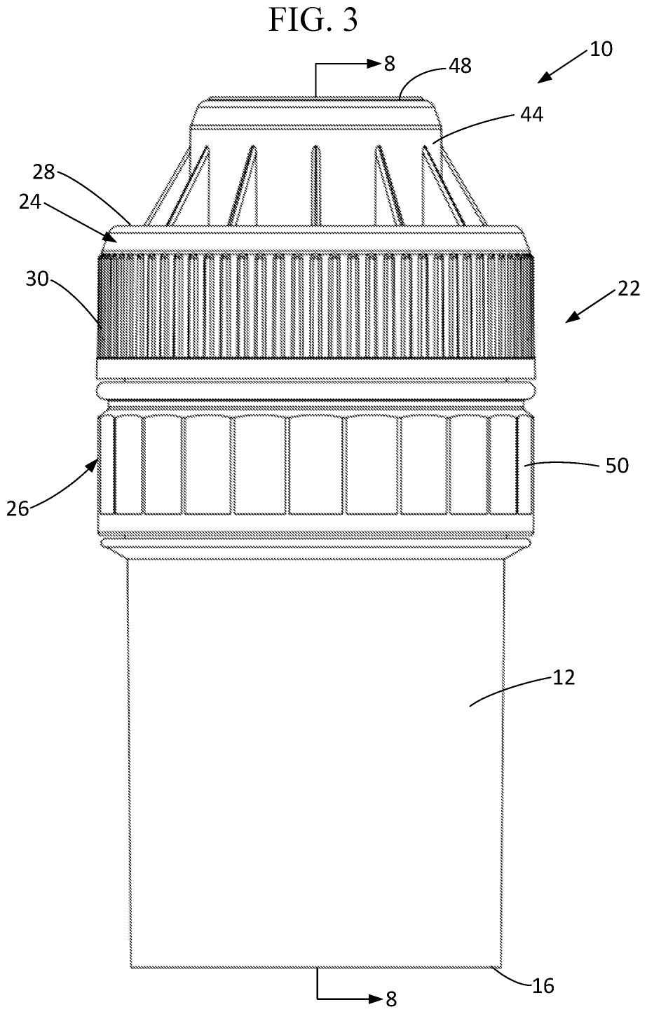

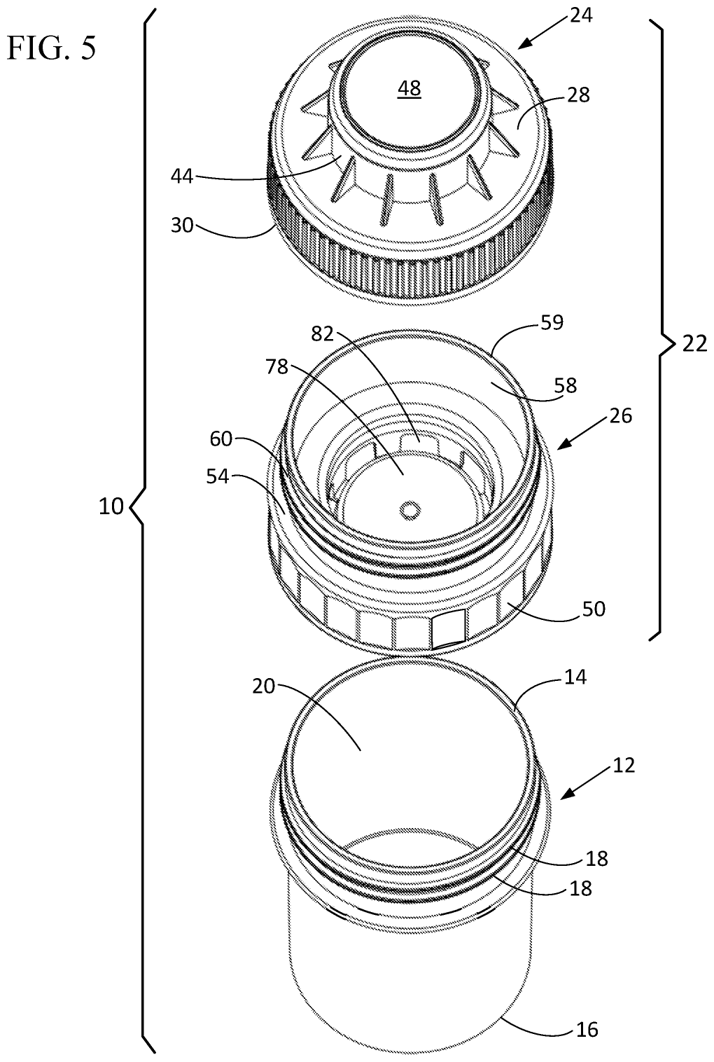

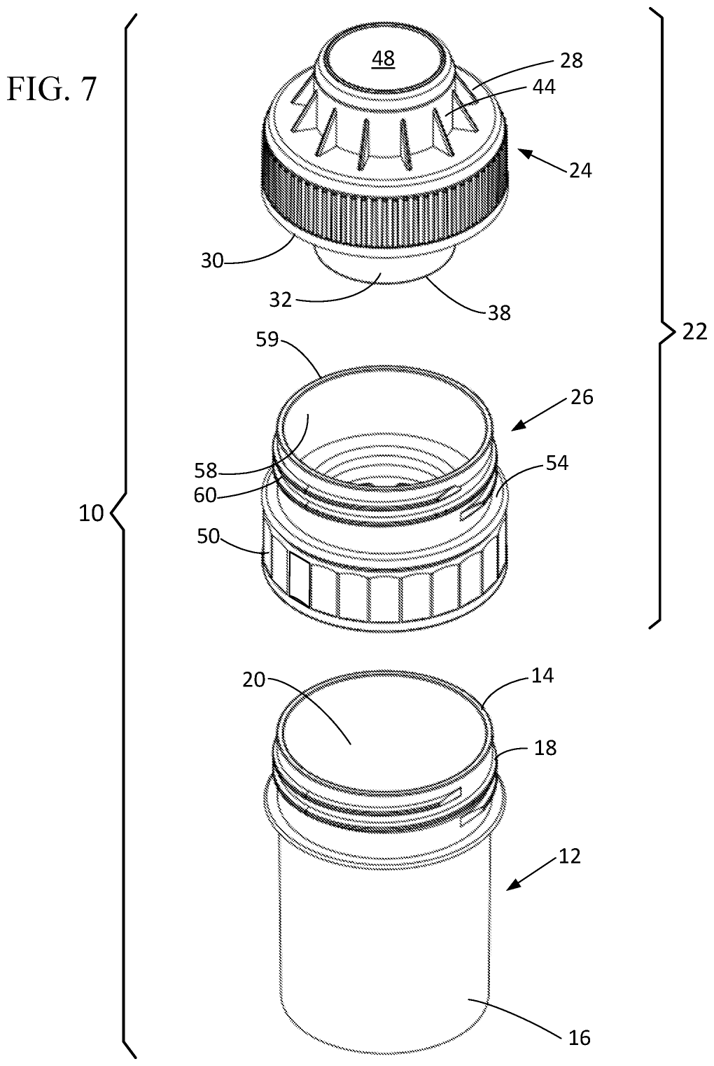

In one embodiment, the container assembly 10 includes a container 12 having an upper end 14 and a lower end 16 (see, e.g., FIGS. 1-3 and 5-9). In one embodiment, the container 12 includes one or more connectors 18 formed on an external surface of the container 12 adjacent the upper end 14. In one embodiment, the connectors 18 include one or more threads. In one embodiment, the container 12 includes a chamber 20 (see, e.g., FIG. 5) for receiving a biological sample or specimen therein for storage and/or transportation.

Referring primarily to FIGS. 1, 5, 6, 8 and 9, the container assembly 10 includes a cap 22 configured to be removably attached to the container 12 and close off the chamber 20 in a liquid-tight manner in accordance with one embodiment. In one embodiment, the cap 22 includes an upper movable section 24 and a lower base section 26 which are removably and/or movably attached to each other. In one embodiment, each of the movable and base sections 24, 26 is a monolithically formed or molded as a single piece. In another embodiment, each of the movable and base sections 24, 26 may be formed in multiple pieces, which are then attached to one another by conventional attachment processes.

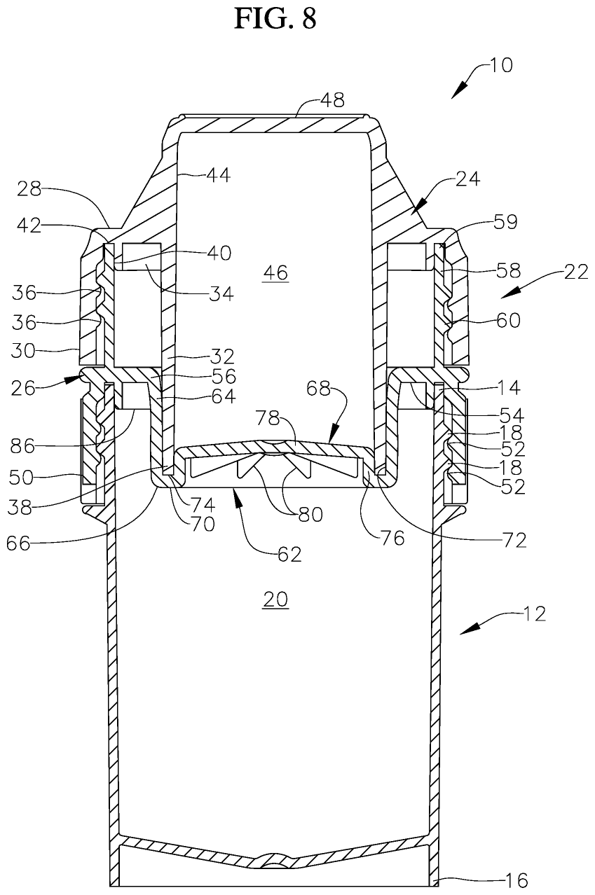

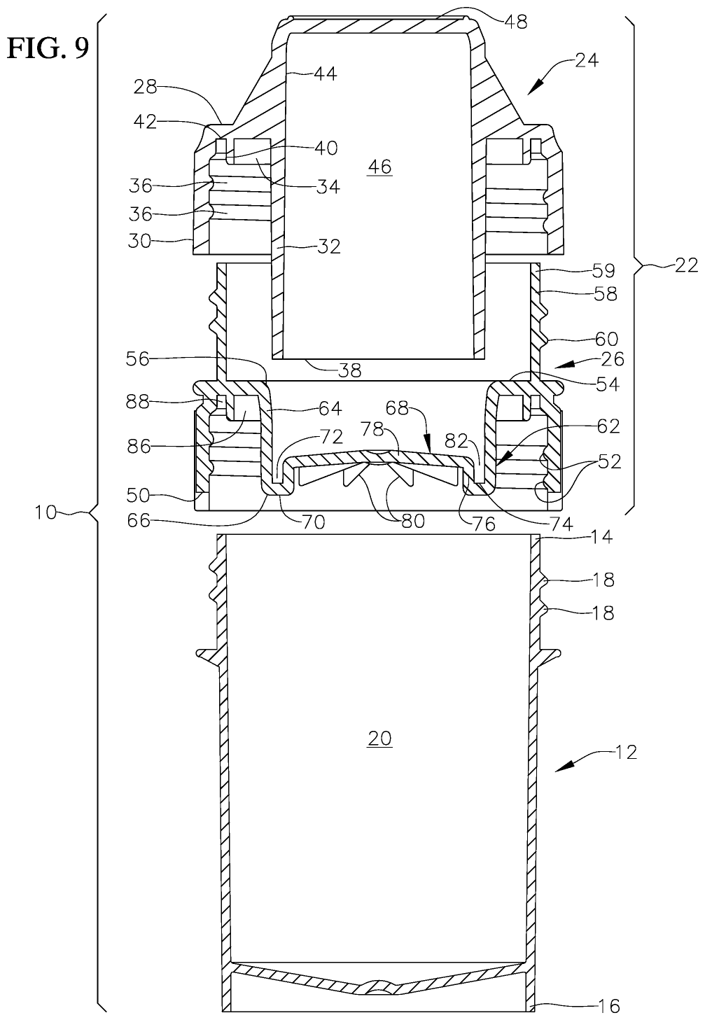

In one embodiment, the movable section 24 has a top 28, as well as a skirt 30, a retaining member 32 and a plug or sealing member 34, all of which depend from the top 28 (see, e.g., FIGS. 1, 5, 6, 8 and 9). In one embodiment, the skirt 30 has one or more connectors 36 formed in an inner surface wall thereof. In one embodiment, the connectors 36 include one or more threads. In one embodiment, the retaining member 32 is in the form of a cylindrical member having an open bottom end 38. In one embodiment, a trough 40 is formed between the skirt 30 and the plug 34, while a seat 42 (see FIGS. 8 and 9) is formed at a top end of the trough 40 for purposes to be discussed below.

In one embodiment, the movable section 24 has a cylindrical projection 44 projecting upwardly from the top 28 (see, e.g., FIGS. 1, 7, 8 and 9). In one embodiment, the cylindrical projection 44 is a continuation of the retaining member 32, as illustrated in FIGS. 8 and 9. In one embodiment, the cylindrical projection 44 and the retaining member 32 define a reservoir chamber 46 in communication with the bottom end 38 of the retaining member 32 for purposes to be discussed hereinbelow. In one embodiment, the reservoir chamber 46 is closed off at its upper end by a top wall 48, which is connected to the cylindrical projection 44.

In one embodiment, the base section 26 of the cap 22 includes a skirt 50 provided with one or more connectors 52 on an inner surface thereof (see, e.g., FIGS. 1, 6, 8 and 9). In one embodiment, the connectors 52 are configured so as to engage the connectors 18 of the container 12. In one embodiment, the connectors 52 include one or more threads configured to mate with the threads of the connectors 18 of the container 12.

In one embodiment, the base section 26 of the cap 22 includes a ledge member 54 extending radially inwardly from the skirt 50, terminating at an inner annular edge 56 (see, e.g., FIGS. 1, 5-8 and 9). In one embodiment, a cylindrical projection 58 projects from the ledge member 54 and is provided with an open top end 59, as well as one or more connectors 60 on an outer surface thereof. In one embodiment, the connectors 60 of the cylindrical projection 58 are configured so as to engage the connectors 36 of the movable section 24. In one embodiment, the connectors 60 include one or more threads configured to mate with the threads of the connectors 36 of the movable section 24.

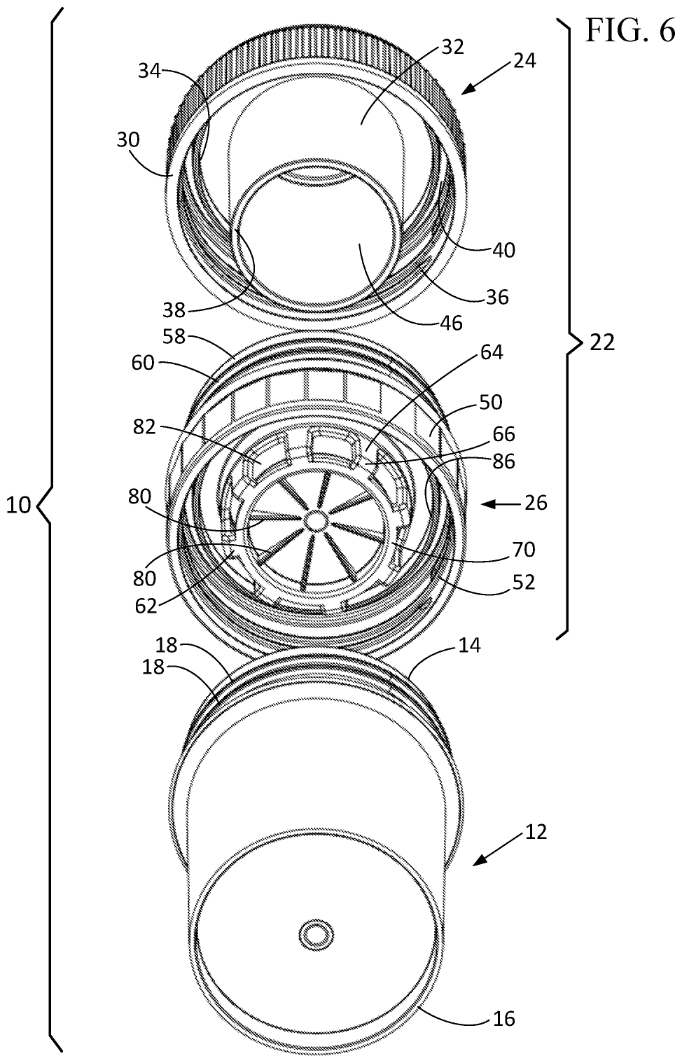

In one embodiment, a retaining member 62 depends from the inner annular edge 56 of the ledge member 54 (see, e.g., FIGS. 6, 8 and 9). More particularly, the retaining member 62 includes a generally cylindrical wall 64 projecting downwardly from the inner annular edge 56 and having a bottom end 66. In one embodiment, the retaining member 62 also includes a plug 68 (see, e.g., FIGS. 8 and 9) located substantially coaxially within the cylindrical wall 64 for purposes to be discussed hereinbelow. In one embodiment, a spacer member 70 (see, e.g., FIGS. 6, 8 and 9) connects the bottom end 66 of the cylindrical wall 64 to the plug 68, forming an annular trough 72 between the plug 68 and the bottom end 66 of the cylindrical wall 64, as well as a seat 74 defining a bottom of the trough 72. In one embodiment, the plug 68 is configured to be inserted into the open bottom end 38 of the retaining member 32 of the movable section 24 for the purpose of closing off the open bottom end 38 in a liquid-tight manner. In one embodiment, the plug 68 includes an annular sealing wall 76 (see, e.g., FIG. 9), which projects generally upwardly from the spacer member 70, and a dome member 78, which is connected to the sealing wall 76 for covering up a space formed within the sealing wall 76. In one embodiment, the plug 68 includes a plurality of reinforcing ribs 80 formed on the underside of the dome member 78 and connected to the sealing wall 76 for reinforcing the plug 68 such that the plug 68 is inhibited from buckling when engaging the bottom end 38 of the retaining member 32 of the movable section 24.

In one embodiment, the retaining member 62 of the base section 26 includes a plurality of discharge openings 82 (see, e.g., FIGS. 5, 6 and 9) formed in the cylindrical wall 64 adjacent the bottom end 66, and optionally extending into the spacer member 70, as illustrated in FIG. 6. In one embodiment, the discharge openings 82 are arranged along the periphery of the cylindrical wall 64 and surround the plug 68 for purposes to be discussed hereinbelow.

In one embodiment, the base section 26 includes a plug or sealing member 86 (see, e.g., FIGS. 6, 8 and 9) depending from the ledge member 54. In one embodiment, the plug 86 cooperates with the skirt 50 so as to form an annular trough 88 (see FIG. 9). In one embodiment, the plug 86 and the trough 88 are configured so as to engage and receive the upper end of 14 of the container 12 for closing or sealing off the chamber 20 of the container 12 when the cap 22 is properly attached to the container 12.

In one embodiment, with the movable and base sections 24, 26 of the cap 22 disassembled from one another, the movable section 24 is turned upside down, providing access to the reservoir chamber 46. In one embodiment, a predetermined amount of a desired solution or liquid is poured into the reservoir chamber 46 through the open bottom end 38 of the retaining member 32. In one embodiment, the desired solution can be a preservation solution for preserving a biopsy tissue sample, such as formalin. With the movable section 24 still in its upside-down position to contain the desired solution in the reservoir chamber 46, the base section 26 is attached to the movable section 24 to close or seal off the open bottom end 38 and hence the reservoir chamber 46 of the movable section 24. In one embodiment, the cylindrical projection 58 of the base section 26 is initially inserted into the skirt 30 and rotated such that the connectors 36 of the skirt 30 engage or mesh with the connectors 60 of the cylindrical projection 58. As the cylindrical projection 58 is rotated into the skirt 30, the open bottom end 38 of the movable section 24 is inserted into the trough 72 and receives the plug 68. Likewise, the open top end 59 of the cylindrical projection 58 is inserted into the trough 40 of the movable section 24 and receives the plug 34. In one embodiment, when the movable section 24 is properly assembled with the base section 26, it is positioned in its closed position, in which the open bottom end 38 of the retaining member 32 and the open top end 59 of the cylindrical projection 58 seat against the seat 70 of the base section 26 and the seat 42 of the movable section 24, respectively (see FIG. 8). In this arrangement, the plug 68 and the plug 34 engage the bottom end 38 of the movable section 24 and the top end 59 of the base section 26, respectively, in a liquid-tight manner such that the reservoir chamber 46 forms a reservoir for containing the desired solution therewithin. In one embodiment, due to the sealing engagement between the bottom end 38 of the movable section 24 and the plug 68 (and optionally with the seat 74) of the base section 26 and between the top end 59 of the base section 26 and the plug 34 (and optionally with the seat 42 and skirt 30) of the movable section 24, the desired solution is prevented from leaking out from the reservoir chamber 46 and the cap 22, respectively. In one embodiment, one or more additional sealing mechanisms, such as an O-ring or other sealing members, may optionally be provided (e.g., on the plug 68, the bottom end 38 of the movable section 24, the spacer member 70, the seat 74, and/or the cylindrical wall 64; and on the plug 34, the top end 59 of the base section 26, the seat 42 and/or the skirt 30 of the movable section 24) to ensure a liquid-tight engagement between the movable and base sections 24, 26 and to prevent leakage of the solution from the reservoir chamber 46 or the cap 22. With the movable and base sections 24, 26 properly assembled with one another, the cap 22 is mounted or screwed onto the upper end 14 of the container 12 to complete the container assembly 10.

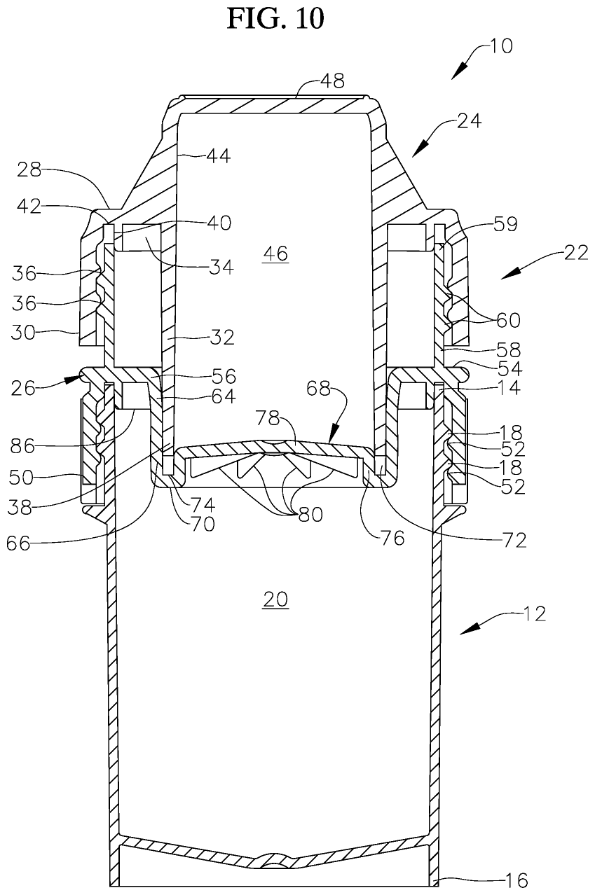

In use, once a biological specimen, such as a biopsy sample, is obtained, it is placed in the container 12 after the entire cap 22 in its assembled state (i.e., with the movable and base sections 24, 26 properly attached with one another) is removed or unscrewed from same. Thereafter, with the movable and base sections 24, 26 still securely attached to each other, the cap 22 is mounted or screwed onto the upper end 14 of the container 12. In one embodiment, when the cap 22 is properly attached to the container 12, the plug 86 of the base section 26 engages the upper end 14 of the container 12 in a liquid-tight manner and thereby closes off the chamber 20 in a liquid-tight manner. At this point, the movable section 24 is in its closed position, in which the sealing engagement between the movable section 24 and the base section 26 blocks off fluid communication between the reservoir (i.e., the reservoir chamber 46) and the discharge openings 82 such that the solution is contained or retained within the reservoir (see FIG. 8). To release the solution to the chamber 20 of the container 12, the movable section 24 of the cap 22 is rotated so as to partially unscrew it from the lower section 26, breaking the sealing engagement between the bottom end 38 of the retaining member 32 of the movable section 24 and the plug 68 of the base section 26. More particularly, the rotation of the movable section 24 causes it to move in an axial direction to its open position, in which the bottom end 38 of the retaining member 32 is disengaged, at least partially, from the plug 68 of the base section 26 (see FIG. 10), thereby allowing fluid communication between the reservoir chamber 46 and the discharge openings 82 and causing discharge of the solution from the reservoir chamber 46 into the chamber 20 of the container 12 through the discharge openings 82.

In one embodiment, the movable section 24 is partially attached to the base section 26 while the solution is released from the reservoir chamber 46 to the chamber 20. In one embodiment, locking or other mechanisms may be provided on the movable and/or base sections 24, 26 so as to prevent complete dismount of the movable section 24 from the base section 26. Once the solution has been released from the reservoir chamber 46 to the chamber 20, the movable section 24 is tightened onto the base section 26 and to provide a sealing engagement between the movable section 24 and the base section 26 as discussed above so as to prevent leakage of fumes from any solution remaining in the cap 22. Because the base section 26 remains securely attached to the container 12 during this process, the solution released into the chamber 20 and its fume are contained within the chamber 20.

In one embodiment, a staining solution or reagent (e.g., eosin, hematoxylin and eosin (H&E) dyes, etc.), is optionally included in the preservation solution (e.g., a formalin solution) that is pre-stored in the reservoir chamber 46. In another embodiment, the preservation solution includes a buffer so as to carry out a fixation of a biopsy sample in a substantially neutral pH. Excess acidity formation may create formalin-heme pigment that appears as black. To neutralize the pH of the preservation solution (e.g., a 10% formalin solution) a buffer is optionally, added. In one embodiment, the buffer includes sodium phosphate, such as sodium phosphate monobasic or dibasic. In one embodiment, the preservation solution may include the following composition:

| Component | Content (w %) |
|---|---|
| Sodium Phosphate, Dibasic | <about 1 |
| Sodium Phosphate Monobasic | <about 1 |
| Water | >about 90 |
| Formalin (37%) | <about 10 |
| Sodium Chloride | >about 0.1 |
| Eosin | <about 0.1 |

In one embodiment, a rinsing and/or cleaning solution is pre-stored in the chamber 20 of the container 12. In one embodiment, the rinsing and/or cleaning solution includes a saline solution. In another embodiment, the rinsing and/or cleaning solution includes a staining solution or reagent (e.g., eosin, hematoxylin and eosin (H&E) dyes, etc.), in a manner similar to that discussed above for the preservation solution. In one embodiment, one or more additional functional solutions, reagents or materials may be pre-stored in the reservoir chamber 46 and/or the chamber 20 of the container assembly 10.

In on embodiment, a biopsy sample can be placed in the container 12 without performing a separate rinsing/cleaning or staining process. More particularly, after the biopsy sample has been placed in the chamber 20 and the cap 22 has been applied to the container 12 to close off the chamber 20, the container assembly 10 is shaken to rinsing and/or clean the biopsy sample with the rinsing/cleaning solution pre-stored in the container 12. The rinsing/cleaning solution may be, or needs not be, discarded after the shaking step. Thereafter, the movable section 24 of the cap 22 is rotated from its closed position to discharge the preservation solution, which is pre-stored in the reservoir chamber 46, into the chamber 20 through the discharge openings 82, as discussed above. Because the preservation solution contains a staining reagent, the biopsy sample is stained automatically without subjecting same to a separate staining process. As a result, because the biopsy sample is rinsed and then stained directly in the chamber 20 of the container 12, the container assembly 10 eliminates the need to perform separate rinsing/cleaning and staining steps prior to placing the biopsy sample in the container 12.

It should be appreciated that the present invention has numerous advantages. For instance, the solution is contained within the reservoir chamber 46 and is discharged directly into the chamber 20 after placement of a biopsy sample, it or its fume is inhibited from coming in contact with a user. In addition, the operation of the container assembly 10 is simplified, i.e., the movable section 24 only needs to be unscrewed slightly and then screwed back on to release the solution into the chamber 20.

It will be understood that the embodiments described herein are merely exemplary and that a person skilled in the art may make many variations and modifications without departing from the spirit and scope of the invention. All such variations and modifications are intended to be included within the scope of the invention, as defined by the appended claims.

We claim:

1. A container assembly for collecting a tissue sample, said container assembly comprising a container having a chamber therein, said chamber being configured to receive the tissue sample; and a cap configured to be attached to said container to close off said chamber, said cap including a base section, which is configured to be attached to said container, and a movable section, which is configured to be movably coupled to said base section, said base section and said movable section being configured to form a reservoir therebetween, said reservoir being configured so as to contain a preservation solution, said base section including at least one opening in fluid communication with said chamber when said cap is attached to said container, said movable section being movable relative to said base section between a closed position, in which said movable section engages said base section so as to block off fluid communication between said reservoir and said at least one opening such that the preservation solution is contained in said reservoir, and an open position, in which said reservoir is in fluid communication with said at least one opening for discharging the preservation solution from said reservoir into said chamber through said at least one opening.

2. The container assembly of claim 1, wherein said base section includes a first retaining member, said at least one opening being formed in said first retaining member; and wherein said movable section includes a second retaining member adapted to engage said first retaining member so as to define said reservoir.

3. The container assembly of claim 2, wherein said second retaining member of said movable section is configured to engage said first retaining member of said base section in a liquid-tight manner so as to contain the preservation solution in said reservoir when said movable section is in its said closed position and to disengage, at least partially, from said first retaining member of said base section so as to allow the preservation solution to discharge from said reservoir to said chamber through said at least one opening when said movable section is in its said open position.

4. The container assembly of claim 3, wherein said second retaining member of said movable section includes a cylindrical wall having an open bottom end; and wherein said first retaining member includes a plug configured to close off said open bottom end of said cylindrical wall of said movable section.

5. The container assembly of claim 4, wherein said cylindrical wall of said movable section and said plug of said base section are configured to define said reservoir, said cylindrical wall of said movable section being configured to engage said plug such that said plug closes off said open bottom end of said cylindrical wall so as to contain the preservation solution in said reservoir when said movable section is in its said closed position, said cylindrical wall being configured to disengage, at least partially, from said plug so as to allow the preservation solution to discharge from said reservoir to said chamber through said at least one opening when said movable section is in its said open position.

6. The container assembly of claim 5, wherein said at least one opening includes a plurality of discharge openings formed in said first retaining member, said discharge openings being arranged so as to surround said plug.

7. The container assembly of claim 1, wherein said movable section is rotatably coupled to said base section, said movable section being movable relative to the base section in an axial direction in response to the rotation of said movable section relative to said base section.

8. The container assembly of claim 7, wherein said movable section is movable in said axial direction away from said base section when said movable section moves from its said closed position to its said open position.

9. The container assembly of claim 8, wherein said base section includes at least one first connector; wherein said movable section includes at least one second connector, said at least one first connector being engageable with said at least one second connector so as to permit rotational movement of said movable section relative to said base section; wherein said base section includes at least one third connector; wherein said container includes at least one fourth connector, said at least one third connector being engageable with said fourth connector so as to movably attach said base section to said container; and wherein each of said first, second, third and fourth connectors includes at least one thread.

10. The container assembly of claim 1, wherein said container includes a rinsing solution in said chamber; and wherein the preservation solution includes a staining reagent.

11. A container assembly for collecting a tissue sample, said container assembly comprising a container having a chamber therein, said chamber being configured to receive the tissue sample; and a cap attached to said container to close off said chamber, said cap including a base section, which is attached to said container, and a movable section, which is movably coupled to said base section, said base section and said movable section forming a reservoir therebetween for containing a preservation solution, said base section including at least one opening in fluid communication with said chamber, said movable section being in a closed position, in which said movable section engages said base section so as to block off fluid communication between said reservoir and said at least one opening such that said preservation solution is contained within said reservoir, and said movable section being movable to an open position, in which said movable section is, at least partially, disengaged from said base section so as to allow fluid communication between said reservoir and said at least one opening for discharging said preservation solution from said reservoir into said chamber through said at least one opening.

12. The container assembly of claim 11, wherein said base section includes a first retaining member, said at least one opening being formed in said first retaining member; wherein said movable section includes a second retaining member adapted to engage said first retaining member; and wherein said first retaining member of the said base section and said second retaining member of said movable section define said reservoir, said second retaining member of said movable section engaging said first retaining member of said base section in a liquid-tight manner so as to contain the preservation solution in said reservoir when said movable section is in its said closed position and disengaging, at least partially, from said first retaining member of said base section so as to allow the preservation solution to discharge from said reservoir to said chamber through said at least one opening when said movable section is in its said open position.

13. The container assembly of claim 12, wherein said second retaining member of said movable section includes a cylindrical wall having an open bottom end; and wherein said first retaining member includes a plug configured to close off said open bottom end of said cylindrical wall of said movable section.

14. The container assembly of claim 13, wherein said movable section is rotatably coupled to said base section, said movable section being movable relative to said base section in an axial direction away from said base section when said movable section moves from its said closed position to its said open position.

15. The container assembly of claim 11, wherein said container includes a rinsing solution in said chamber; and wherein the preservation solution includes a staining reagent.

16. A method for collecting a tissue sample, comprising the steps of obtaining in a container having a chamber therein; placing the tissue sample in the chamber of the container; obtaining a cap including a base section, which includes at least one opening, and a movable section, which is movably coupled to the base section, the movable section being in a closed position, in which the movable section engages the base section and form a reservoir containing a preservation solution; placing the cap on the container so as to close off the chamber of the container, the at least one opening being in fluid communication with the chamber; moving the movable section to an open position, in which the movable section is, at least partially, disengaged from the base section so as to allow fluid communication between the reservoir and the at least one opening such that the preservation solution is discharged from the reservoir into the chamber through the at least one opening.

17. The method of claim 16, wherein the base section includes a first retaining member, the at least one opening being formed in the first retaining member; wherein the movable section includes a second retaining member adapted to engage the first retaining member; and wherein the first retaining member of the base section and the second retaining member of the movable section define the reservoir, the second retaining member of the movable section engaging the first retaining member of the base section in a liquid-tight manner so as to contain the preservation solution in the reservoir when the movable section is in its said closed position and disengaging, at least partially, from the first retaining member of the base section so as to allow the preservation solution to discharge from the reservoir to the chamber through the at least one opening when the movable section is in its said open position.

18. The method of claim 17, wherein the second retaining member of the movable section includes a cylindrical wall having an open bottom end; and wherein the first retaining member includes a plug configured to close off the open bottom end of the cylindrical wall of the movable section.

19. The method of claim 18, wherein the movable section is rotatably coupled to the base section, the movable section being movable relative to the base section in an axial direction away from the base section when the movable section moves from its said closed position to its said open position.

20. The method of claim 16, wherein said container includes a rinsing solution in said chamber such that the tissue sample can be rinsed with the rinsing solution when the tissue sample is placed in the chamber of the container; and wherein the preservation solution includes a staining reagent such that the tissue sample can be strained by the preservation solution when the preservation solution is discharged from the reservoir into the chamber through the at least one opening.
